(19) United States
(12) Patent Application Publication
(10) Pub. No.: US 2007/0264346 A1
(43) Pub. Date: Nov. 15, 2007

(54) Multimicroparticulate pharmaceutical forms for oral administration

(75) Inventors: Florence Guimberteau, Montussan (FR); Frederic Dargelas, Lyon (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/707,054

(22) Filed: Feb. 16, 2007

Related U.S. Application Data
(60) Provisional application No. 60/773,657, filed on Feb. 16, 2006.

(51) Int. Cl. A61K 9/26

(52) U.S. Cl. 424/488; 424/484

(57) ABSTRACT

The object of the present invention is to minimize the risks of dose dumping associated with the concomitant consumption of alcohol and certain modified-release pharmaceutical or dietetic forms. The invention relates to an oral form comprising microparticles of the reservoir type for the modified release of at least one active principle (AP), characterized in that it is resistant to immediate dumping of the dose of AP in the presence of alcohol. In particular, the oral form according to the invention is characterized in that the time taken to release 50% of the AP in an alcoholic solution is not reduced more than 3-fold relative to the time taken to release 50% of the AP in an alcohol-free aqueous medium. The form comprises an agent D, which is a pharmaceutically acceptable compound whose hydration or solvation rate or capacity is greater in an alcohol-free aqueous medium than in alcoholic solution

### FIELD OF THE INVENTION

The present invention relates to the field of pharmaceutical or dietetic forms for the modified release of medicinal active principles (AP) intended for oral administration.

The present invention relates to forms for oral administration which contain at least one AP and are capable of maintaining a modified release of the AP in an alcoholic solution, i.e. they are not subject to dose dumping in the presence of alcohol. Preferably, the invention relates to modified-release pharmaceutical forms whose release profile is not significantly affected in alcoholic solution.

The present invention relates more particularly to forms of the type referred to in the previous paragraph which comprise a plurality of reservoir microparticles.

The present invention relates even more particularly to pharmaceutical forms for which the ingestion of alcohol during administration is not recommended.

The invention further relates to a process for the preparation of the pharmaceutical forms defined above.

### CONTEXT OF THE INVENTION

The value of modified-release pharmaceutical forms for the administration of a drug is well know. In particular, they provide a better cover of the therapeutic need since the useful plasma AP concentration can be maintained longer than in the case of instantaneous-release forms. In addition, they make it possible to avoid or limit the magnitude and number of peaks of excessive plasma AP concentration, thereby reducing the toxicity of the drug and its side effects. Furthermore, by virtue of their increased duration of action, these systems make it possible to limit the number of daily dosage units, thus reducing constraint for the patient and improving compliance with the treatment.

Systems have thus been sought which make it possible to prolong the action of a drug, and this objective is the subject of numerous references. The work by Buri, Puisieux, Doelker and Benoft entitled Formes Pharmaceutiques Nouvelles (New Pharmaceutical Forms), Lavoisier 1985, pp 175-227, may be consulted in this regard.

Modified-release (MR) forms have been developed, particularly for AP which have a narrow therapeutic window, i.e. whose effective doses are similar to those at which undesirable effects can manifest themselves, in order to clip the plasma peak (Cmax), the objective being to maintain plasma concentrations for a prolonged period at values below those at which there is a risk of undesirable effects.

Such forms have also been developed for allowing a more stable and continuous impregnation of the organism with AP without the subject needing to increase the number of dosage units. Thus there are forms which contain, in one dosage unit, the amount of AP required for 24 h of treatment, this form of course being intended for administration only once a day.

Modified-release pharmaceutical forms include systems in which the release of the AP is controlled by a coating enveloping the AP, these systems also being called reservoir systems. In another group, called matrix systems, the AP, intimately dispersed in a matrix based e.g. on a polymer, is released from the tablet by diffusion and erosion.

Numerous studies have been conducted to ensure that the release of the AP is effectively controlled so as to avoid the massive overdosing which would result from an accidentally immediate release of the amount of AP intended for prolonged release. This control is extremely important in practice because it is active products with a narrow therapeutic window which most frequently benefit from the modified release technique. In this case said accidental immediate release (dose dumping) would have exactly opposite effects to those which the technique used was attempting to achieve.

Dose dumping can occur e.g. in the case of a monolithic matrix tablet which the patient chews before swallowing, short-circuiting a slow disintegration step in the stomach. An advantageous way of avoiding the risk associated with chewing consists in preparing a microparticulate form in which each microparticle possesses the properties of modified release.

The use of multi(micro)particulate forms limits the risk of massive release and makes it possible to reduce the interindividual and intraindividual variability associated with gastric emptying.

PCT application WO-A-96/11675 describes modified-release micro-capsules capsules for the oral administration of medicinal and/or nutritional active principles (AP), their size being less than or equal to 1000 μm. These microcapsules consist of particles covered with a coating material consisting of a mixture of a film-forming polymer (ethyl cellulose), a hydrophobic plasticizer (castor oil), a surfactant and/or lubricant (magnesium stearate) and a nitrogen-containing polymer (polyvinylpyrrolidone: povidone, PVP). These microcapsules are also characterized by their ability to reside in the small intestine for a long time (at least 5 h) and by their ability, during this residence time, to allow the absorption of the AP over a period longer than the natural transit time in the small intestine.

PCT application WO-A-03/030878 describes a multimicrocapsular oral pharmaceutical form in which the release of the AP is governed by a dual release triggering mechanism: “triggering time” and “triggering pH”. This form consists of microcapsules (200 to 600 μm) comprising a core that contains the AP and is covered with a coating (maximum 40% by weight) comprising a hydrophilic polymer A carrying functional groups ionized at neutral pH (Eudragit® L) and a hydrophobic compound B (vegetable wax melting at 40-90° C.), where B/A is between 0.2 and 1.5.

In addition to the essential constituents A and B, the microcapsule coating can comprise other conventional ingredients such as, in particular:
colorants;
plasticizers, e.g. dibutyl sebacate;
hydrophilic compounds, e.g. cellulose and derivatives thereof or polyvinyl-pyrrolidone and derivatives thereof;
and mixtures thereof.

These examples illustrate the efforts made to avoid the failure of the different AP modified-release systems.

However, it has recently become apparent that, despite these efforts, the bulk of the AP can be released too rapidly when the MR pharmaceutical form is ingested concomitantly with alcohol.

Thus, in the USA in October 2005, the Food and Drug Administration expressed the idea that a study of the resistance of MR forms to the dose dumping potentially induced by alcohol would be worth conducting for drugs.

In fact, recent studies have shown that the presence of alcohol can accelerate the release of an AP contained in an MR pharmaceutical form. In a first analysis, this alcohol effect can be explained by a degradation of the modified-release system or by a modification of the solubility of the AP in the presence of a significant amount of alcohol. This situation is all the more likely to be encountered—and the consequences are likely to be all the more serious—if a large amount of alcoholic drink is ingested, if the drink has a high alcoholic strength and if the subject has an empty stomach. In fact, in this last case, the stomach will essentially contain the ingested drink mixed with a small amount of gastric juice. In practice, therefore, the ingestion of alcohol concomitantly with the administration of an MR pharmaceutical form can result in the accelerated and potentially dangerous release of the AP in the patient. Depending on the type of AP, this accelerated release of the AP at best renders the MR pharmaceutical form totally ineffective, and at worst jeopardizes the patient's vital prognosis.

This harmful acceleration of the release can result in a loss of activity of the drug, as would be the case, for example, of proton pump inhibitors, whose excessively early release in an acidic gastric medium would lead to their degradation and hence to the inefficacy of the treatment.

Conversely, a more dangerous case is that of certain tranquilizers, antidepressants or opiate analgesics, where it is the vital prognosis which would be in question because of the seriousness of the side effects following an overdose.

One particular group of drugs for which a massive release of the AP would be particularly harmful is the group of products which have an unfavorable pharmacological interaction with alcohol, an incompatibility or an exacerbation of the side effects.
Thus, for example, an undesirable effect of the opiate analgesic group of drugs is that they are capable of inducing respiratory depression; this can be aggravated by the concomitant consumption of alcohol because of the false routes and the swallowing pneumopathies conventionally caused by alcohol abuse.
Likewise, very widely used drugs such as tranquilizers and antidepressants have effects on the central nervous system (loss of vigilance, risks of somnolence) which are exacerbated by the simultaneous consumption of alcohol.
Interactions of alcohol with antihistamines (potentiation of the sedative effect, somnolence and loss of attention, giddiness) and with non-steroidal anti-inflammatories or NSAI (potentiation of the risk of digestive bleeding) may also be mentioned.

In the case of monolithic matrix forms, accidental dose dumping results in very high concentrations of AP in the digestive system, where the form is located, and this can cause lesions.

The problem of dose dumping in the presence of alcohol has not yet been solved satisfactorily, particularly in the case of multimicroparticulate forms. In particular, there is a need for a modified-release multi microparticulate pharmaceutical form for the oral administration of AP which is capable of maintaining the modified release of the AP in an alcoholic solution, i.e. whose AP release profile is not accelerated at the risk of compromising a patient's vital prognosis, and preferably whose AP release profile is not significantly affected in alcoholic solution.

### OBJECTS OF THE INVENTION

One essential object of the present invention is to propose a multimicro-particulate pharmaceutical form for the modified release of at least one medicinal or dietetic active principle (AP) which is intended for oral administration and makes it possible to avoid or limit the dose dumping induced by the consumption of alcohol during the administration of said pharmaceutical form, thereby affording greater therapeutic safety and better efficacy.

Another essential object of the present invention is to propose a multimicroparticulate pharmaceutical form for the modified release of at least one AP which is intended for oral administration and for which the release of the AP is not significantly affected by the presence of alcohol.

The microparticles according to the invention are optionally capable of being processed to tablets, sachets, capsules, suspensions to be taken orally, etc.

Another essential object of the present invention is to propose a multimicroparticulate pharmaceutical form for the modified release of at least one AP which is intended for oral administration and for which the in vitro AP release profile in the ethanol-free dissolution media conventionally used is similar to the profile obtained in the same media to which ethanol has been added.

Another essential object of the present invention is to propose a multimicroparticulate pharmaceutical form for the modified release of at least one AP which is intended for oral administration and which has an in vitro release profile in the presence of ethanol that does not compromise a patient's vital prognosis.

Another essential object of the present invention is to provide a multimicro-particulate pharmaceutical form for the modified release of at least one AP which is intended for oral administration and which represents an improvement relative to the forms described in international patent applications WO-A-96111675 and WO-A-03/03878, particularly in respect of the behavior in alcoholic solution.

Another essential object of the present invention is to propose a process for obtaining a multimicroparticulate pharmaceutical form for the modified release of at least one AP which is intended for oral administration and whose in vitro AP release profile is not significantly affected in alcoholic solution, or at least whose release is not accelerated at the risk of compromising the patient's vital prognosis.

Definitions

In terms of the present disclosure of the invention:
the abbreviation “AP” denotes both a single active principle and a mixture of several active principles. The AP can be in the free form or in the form of a salt, an ester, a hydrate, a solvate, a polymorph, isomers or other pharmaceutically acceptable forms;
the ingested alcohol can originate from different alcoholic drinks or beverages, such as beer, wine, cocktails, spirits or mixtures thereof;
in vitro the term “alcohol” represents ethanol and the term “alcoholic solution” or “alcoholic medium” represents an aqueous solution of ethanol;
the term “hypromellose” represents hydroxypropyl methyl cellulose or HPMC;
“reservoir microparticles” denotes microparticles comprising AP which are individually covered with at least one coating allowing the modified release of the AP;
“microparticle” arbitrarily denotes reservoir microparticles and/or microparticles comprising AP that are not necessarily coated;
the in vitro dissolution profiles are prepared as instructed in the European Pharmacopoeia (5th edition, §2.9.3), which describes the dissolution media conventionally used. To simulate the gastric medium of a subject who has absorbed a large amount of alcohol, the dissolution medium is modified by adding ethanol (qsp 20% to 40% by volume);
the abbreviation “MR” denotes modified release;
the term “modified release” denotes that the in vitro release of the AP is such that 75% of the AP is released over a period of more than 0.75 h, preferably of more than 1 h and particularly preferably of more than 1.5 h. A modified-release pharmaceutical form can comprise e.g. an immediate-release phase and a slow-release phase. The modified release can be especially a prolonged and/or delayed release. Modified-release pharmaceutical forms are well known in this field; cf., for example, Remington: The science and practice of pharmacy, 19th edition, Mack Publishing Co., Pennsylvania, USA;
“immediate release” means that the release is not of the modified type and denotes the release, by an immediate-release form, of the major part of the AP over a relatively short period, e.g. at least 75% of the AP is released in 0.75 h, preferably in 30 min;
the multimicroparticulate oral pharmaceutical forms according to the invention consist of numerous microparticles whose size is less than a millimeter. Unless indicated otherwise, the diameters of microparticles referred to in the present disclosure are volume-average diameters. These multimicroparticulate forms can be converted to monolithic oral pharmaceutical forms such as tablets, capsules, sachets and reconstitutable suspensions;
the similarity between two dissolution profiles is evaluated using the similarity factor f₂ as defined in the document “Qualité des produits à libération modifiée” (“Quality of modified-release products”) of the Europem Drug Evaluation Agency, document reference CPMP/QWP/604/96 (Annex 3). An f₂ value of between 50 and 100 indicates that the two dissolution profiles are similar,
“dose dumping” is understood as meaning an immediate and unwanted release of the dose after oral ingestion.

### BRIEF DESCRIPTION OF THE INVENTION

It is to the inventors' credit to have found a formulation which makes it possible to eliminate or reduce the modifications in AP release profiles observed in alcoholic solution.

The present inventors have developed MR pharmaceutical forms which have a resistance to alcohol-induced dose dumping. This advantageous property can be demonstrated in particular under conditions that reproduce the physicochemical characteristics expected in vivo. Binge drinking, a form of alcoholism characterized by bouts of high consumption, typically at the end of the week, alternating with long periods of abstinence or moderation, has become an increasingly widespread social activity in certain spheres, and there has been a parallel increase in the risk represented by an accidental release of the dose of AP contained in an MR pharmaceutical form in a subject who has also ingested a large amount of alcohol.

The inventors have studied the sensitivity of various MR pharmaceutical forms in the presence of alcohol. The approach chosen for measuring the resistance of the MR pharmaceutical forms to alcohol-induced dose dumping consists in modifying the conventional dissolution tests for MR pharmaceutical forms by introducing ethanol into the dissolution medium, e.g. at a concentration of 20% or 40% (v/v). The order of magnitude of the final volume is 50 to 900 ml.

For a number of MR phamaceutical forms, it is observed that the co-administration of said form with alcoholic beverages leads to an unwanted acceleration of the release of the AP. To solve this problem, the present invention relates to a novel multimicroparticulate pharmaceutical form for the modified release of at least one medicinal AP which is intended for oral administration, characterized in that it is capable of maintaining the modified release of the AP in an alcoholic solution and, preferably, in that the release profile is not significantly affected in alcoholic solution.

More precisely, the present invention relates to an oral pharmaceutical form comprising microparticles of the reservoir type for the modified release of at least one AP, said form being resistant to immediate dumping of the dose of AP in the presence of alcohol.

Preferably, the oral pharmaceutical form according to the invention, which comprises microparticles of the reservoir type for the modified release of at least one AP both in aqueous dissolution media and in alcoholic solutions, is characterized in that the time taken to release 50% of the AP in alcoholic solution:
is not reduced more than 3-fold relative to the time taken to release 50% of the AP in an alcohol-ee aqueous medium;
is preferably not reduced more than 2-fold relative to the time taken to release 50% of the AP in an alcohol-free aqueous medium;
is preferably not reduced more than 1.5-fold relative to the time taken to release 50% of the AP in an alcohol-free aqueous medium;
is preferably similar to the time taken in an aqueous medium according to the similarity factor f₂ defined above;
or is longer than the time taken to release 50% of the AP in an alcohol-free aqueous medium.

This pharmaceutical form according to the invention comprises micro-particles of the reservoir type and at least one agent D, which is a pharmaceutically acceptable compound whose hydration or solvation rate or capacity is greater in an alcohol-free aqueous medium than in alcoholic solution. The reservoir micro-particles have a mean diameter preferably of less than 2000 μm, particularly preferably of between 50 and 800 μm and very particularly preferably of between 100 and 600 μm. Also, the reservoir microparticles individually consist of a core which comprises the AP and is covered with a coating comprising:
at least one polymer A that is insoluble in the fluids of the digestive tract;
at least one plasticizer B;
and optionally at least one surfactant C.

### BRIEF DESCRIPTION OF THE FIGURES

FIG. 1: Schematic representation of the structure of a coated microparticle.

FIG. 2: Schematic representation of the structure of a coated microparticle.

FIG. 3: Schematic representation of the structure of a pellet or granule comprising microparticles and agent D as binder.

FIG. 4: Schematic representation of a coated tablet containing microparticles.

FIG. 5: Schematic representation of a capsule covered with a coating based on agent D, said capsule containing microparticles.

FIG. 6: Dissolution of the acyclovir capsules prepared in Example 1.

FIG. 7: Dissolution of the metformin capsules prepared in Example 2.

FIG. 8: Dissolution of the acyclovir capsules prepared in Example 3.

FIG. 9: Dissolution of the metformin capsules prepared in Example 4.

FIG. 10: Behavior of sodium starch glycolate (Primojel®/Avebe) in water (FIG. 10A) and in an alcoholic solution (FIG. 10B) after a contact time of 15 min.

FIG. 11: Behavior of guar gum (Grindsted® Guar/Danisco) in water (FIG. 11A) and in an alcoholic solution (FIG. 11B) after a contact time of 15 min.

FIG. 12: Behavior of hydroxypropyl methyl cellulose (Methocel® ES/Dow) in water (FIG. 12A) and in an alcoholic solution (FIG. 12B) after a contact time of 30 min.

FIG. 13: Dissolution of the metformin capsules prepared in Example 6.

### DETAILED DESCRIPTION OF THE INVENTION

The oral pharmaceutical or dietetic form according to the invention comprises microparticles of the reservoir type and allows the modified release of the AP both in aqueous dissolution media and in alcoholic solutions. This form according to the invention is multimicroparticulate, i.e. it comprises, inter alia, reservoir microparticles with a coated or film-coated core comprising the AP. This AP core, or AP microparticle, can be:
crude (pure) AP in pulverulent form, and/or
a matrix granule of AP mixed with various other ingredients, and/or
a supported granule, such as an inert support of e.g. cellulose or sugar, covered with at least one layer containing AP.

In the case of a matrix granule, the matrix contains the AP and optionally other pharmaceutically acceptable excipients such as binders, surfacants, disintegrants, fillers, and agents for controlling or modifying the pH (buffers).

In the case of a supported granule, the inert support can be composed of sucrose and/or dextrose and/or lactose and/or a sucrose/starch mixture. The inert support can also be a cellulose microsphere or any other particle of pharmaceutically acceptable excipient. Advantageously, the inert support has a mean diameter of between 1 and 800 μm, preferably of between 20 and 500 μm.

Apart from the AP, the active layer can optionally contain one or more pharmaceutically acceptable excipients such as binders, surfactants, disintegrants, fillers, and agents for controlling or modifying the pH (buffers).

The form according to the invention can comprise AP microparticles other than reservoir particles, a possible example being microparticles for the immediate release of AP. The latter can be e.g. uncoated AP microparticles of the same type as those useful in the preparation of the reservoir microparticles according to the invention and comprising one or more AP.

In addition, the group of microparticles (reservoir microparticles and/or uncoated microparticles) constituting the form according to the invention can be made up of different populations of microparticles, these populations differing from one another at least in the nature of the AP contained in said microparticles and/or in the composition of the coating and/or the thickness of the coating.

In a first embodiment, at least some of the microparticles for the modified release of AP each contain an AP microparticle covered with at least one coating allowing the modified release of the AP.

Preferably, the AP microparticle is a granule comprising the AP and one or more pharmaceutically acceptable excipients.

In a second embodiment, at least some of the microparticles for the modified release of AP each contain an inert support, at least one active layer comprising the AP and coating the inert support, and at least one coating allowing the modified release of the AP.

As noted above, the reservoir microparticles individually consist of a core which comprises the AP and is covered with a coating. The coating governs the modified release of the AP. It comprises:
at least one polymer A that is insoluble in the fluids of the digestive tract;
at least one plasticizer B;
and optionally at least one surfactant C.

The coating of the reservoir microparticles contains a polymer A that is insoluble in the fluids of the digestive tract, in an amount of 70% to 95%0, preferably of 75% to 95% and particularly preferably of 80% to 95% of the weight of the coating excluding agent D. The polymer A is preferably selected from the q p following group of products:
water-insoluble cellulose derivatives,
(meth)acrlic (co)polymer derivatives,
and mixtures thereof.

Particularly preferably, the polymer A is selected from the following group of products: ethyl cellulose, cellulose acetate-butyrate, cellulose acetate, type A and type B ammonio-methacrylate copolymers (Eudragit® RS, Eudragit® RL, Eudragit® RS PO, Eudragit® PL PO), poly(meth)acrylic acid esters (Eudragit® NE 30D) and mixtures thereof, ethyl cellulose and/or cellulose acetate being particularly preferred.

The plasticizer B is present in the coating of the reservoir microparticles in an amount of 1% to 30% w/w, preferably of 2% to 25% w/w and particularly preferably of 5% to 20% by weight of the coating excluding agent D. The plasticizer B is selected especially from the following group of products:
glycerol and esters thereof, preferably from the following subgroup: acetylated glycerides, glyceryl monostearate, glyceryl triacetate, glyceryl tnbutyrate,
phthalates, preferably from the following subgroup: dibutyl phthalate, diethyl phthalate, dimethyl phthalate, dioctyl phthalate,
citrates, preferably from the following subgroup: acetylcitric acid tributyl ester, acetylcitric acid triethyl ester, tributyl citrate, triethyl citrate,
sebacates, preferably from the following subgroup: diethyl sebacate, dibutyl sebacate,
adipates,
azelates,
benzoates,
chlorobutanol,
polyethylene glycols,
vegetable oils,
fumarates, preferably diethyl fumarate,
malates, preferably diethyl malate,
oxalates, preferably diethyl oxalate,
succinates, preferably dibutyl succinate,
butyrates,
cetyl alcohol esters,
malonates, preferably diethyl malonate,
castor oil (this being particularly preferred),
and mixtures thereof.

The surfactant C is present in the coating of the reservoir microparticles in an amount of 0 to 30% w/w, preferably of 0 to 20% w/w and particularly preferably of 5 to 15% by weight of the coating excluding agent D. The surfactant C is preferably selected from the following group of products:
alkali metal or alkaline earth metal salts of fatty acids, sodium dodecylsulfate and sodium docusate being preferred,
polyoxyethylenated oils, preferably polyoxyethylenated hydrogenated castor oil,
polyoxyethylenelpolyoxypropylene copolymers,
polyoxyethylenated sorbitan esters,
polyoxyethylenated castor oil derivatives,
stearates, preferably calcium, magnesium, aluminum or zinc stearate,
polysorbates,
stearylfumarates, preferably sodium stearylfumarates,
glycerol behenate,
benzalkonium chloride,
acetyltrimethylammonium bromide,
and mixtures thereof

The monolayer or multilayer coating can comprise various other additional adjuvants conventionally used in the field of coating, possible examples being pigments, colorants, fillers, antifoams, etc.

In one particular embodiment of the invention, the coating governing the modified release of the AP by the reservoir microparticles consists of a single layer or a single film coating. This simplifies their preparation and limits the coating rate.

Advantageously, the coating has a sufficient mechanical strength to avoid tearing and/or bursting in the organism until the release of the AP has ended. This ability of the coating to preserve its physical integrity even after complete elution of the AP is observed in particular for coating thicknesses of between 2 μm and 100 μm, i.e. coating rates (weight of the coating, excluding agent D, over total weight of the microparticle) of between 3 and 85%.

It is important to note that the functionality of alcohol resistance is not acquired at the expense of the other specifications demanded for a modified-release pharmaceutical form. In particular, the pharmaceutical form according to the invention can be adapted to a large number of AP having a very wide variety of solubilities in water, e.g. of between a few hundredths of a milligram per liter and a few hundred grams per liter.

Furthermore, the pharmaceutical form according to the invention makes it possible to adjust the release of the AP over a very wide variety of periods, e.g. of between 1 h and 30 h, preferably of between 2 h and 16 h. It is within the understanding of those skilled in the art to adjust the release time by varying the composition and/or thickness of the coating, in particular, and/or the mean size of the microparticles.

The agent D is a pharmaceutically acceptable compound whose hydration or solvation rate or capacity is greater in an alcohol-free aqueous medium than in alcoholic solution. It can be:
a compound whose solubilization rate is greater in water than in alcoholic solution;
a compound that is soluble in water and insoluble in alcoholic solution;
or a compound that is insoluble in water and alcoholic solution and swells more, or more rapidly, in water than in alcoholic solution.

Preferably, the agent D is selected from the following group of products:
crosslinked carboxyalkyl celluloses: crosslinked carboxymethyl celluloses (e.g. sodium croscarmellose),
polyalkylene oxides (e.g. polyethylene oxide or polypropylene oxide),
(hydroxy)(allyl) celluloses (e.g. hydroxypropyl cellulose, hypromellose (or HPMC]),
carboxyalkyl celluloses (e.g. carboxymethyl cellulose) and salts thereof,
celluloses (powdered or microcrystalline),
polacrilin potassium,
polysaccharides, e.g.:
   native starches (e.g. maize, wheat or potato starch) or modified starches (e.g. modified with sodium glycolate),
   alginates and salts thereof such as sodium alginate,
   guar gums,
   carrageenans,
   pectins,
   chitosans and derivatives thereof,
   and mixtures thereof,
proteins, e.g.:
   gelatin,
   albumins,
   casein,
   lactoglobulins,
   and mixtures thereof,
clays such as bentonite, laponite,
and mixtures thereof.

Particularly preferably, the agent D is selected from the following group of
products:
hydroxyalkyl celluloses (e.g. hydroxypropyl cellulose, hypromellose [or HPMC]),
guar gums,
carrageenans,
pullulans,
and mixtures thereof

The agent D can be incorporated in different ways, optionally combined with one another, into the pharmaceutical form according to the invention. It can be:
one of the constituents of the AP core (or uncoated AP microparticle), i.e.:
   in the inert support of the microparticles,
   and/or in the layer containing the AP, deposited on the inert support of the. microparticles,
   and/or in the granules containing the AP;
and/or one of the constituents of the coating of the microparticles;
and/or mixed with the microparticles;
and/or one of the external constituents of a monolithic form (e.g. constituent of a capsule, coating of a tablet or capsule).

In a first embodiment of the invention, the agent D is present in the AP core or uncoated AP microparticle. Preferably, the agent D is present in the core of the microparticles in an amount of 5 to 70%, preferably of 15% to 60%, of the total weight of the AP core.

In a second embodiment of the invention, the agent D is in the coating of the microparticles. In this case, the agent D can alone constitute a coating layer inside or outside the coating that controls the diffusion. It can also be mixed with constituents A, B and optionally C of the coating that governs the modified release of the AP. Preferably, the agent D is present in the coating in an amount of 3 to 30%, preferably of 10% to 20%, of the total weight of the coating. The following compounds are chosen by way of preference: the polymer A is ethyl cellulose, the plasticizer B is castor oil, the surctant is polysorbate and the agent D is selected from guar gum, hypromellose [or HPMC], sodium carboxymethyl cellulose, pullulan, starch glycolate and mixtures thereof.

In a third embodiment, the agent D is included in the binder phase of granules or pellets or else of tablets including the microparticles. The granules, pellets or tablets are obtained by the techniques known to those skilled in the art, e.g. granulation, extrusion or compression. The agent D is present in a mixture with the microparticles in an amount of 2 to 30% w/w, preferably of 5% to 25% w/w and particularly preferably of 5% to 20% w/w, based on the total weight of the mixture.

In a fourth embodiment, the agent D is one of the components of the material constituting the capsule containing the microparticles. For example, the capsule is in the form of a capsule based on an agent D, preferably based on pullulan, hypromellose [or HPMC] or a mixture thereof

In a fifth embodiment, the agent D is in a coating deposited on the capsule containing the microparticles or on the tablet containing the microparticles. For example, the capsule is based on gelatin and the coating contains sodium carboxymethyl cellulose as agent D, preferably in an amount of 25% w/w of sodium carboxymethyl cellulose, based on the weight of the empty capsules.

In case of the fourth and fifth embodiments, a finishing layer may be deposited on the capsule or tablet

As far as the agent D is concerned, the five embodiments can be combined with one another. It is also possible to incorporate different agents D for each of the embodiments.

Preferably, the form according to the invention is made up of one galenical unit or several identical galenical units (e.g. tablet, capsule or sachet) each containing the microparticles.

The form according to the invention can also be a multidose oral suspension that is reconstituted from powder and water before administration.

The form according to the invention can also be a capsule containing a tablet, said tablet containing reservoir microparticles of AP; the tablet can contain one or more agents D and the capsule can be coated with one or more agents D.

Advantageously, the form containing the microparticles for the modified release of AP also comprises conventional, pharmaceutically acceptable excipients, e.g. ones which are usefull for presenting the microparticles in tablet form. These excipients can be the following in particular:
compression aids such as microcrystalline cellulose or mannitol,
colorants,
disintegrants,
flow promoters such as talcum or colloidal silica,
lubricants such as glycerol behenate or stearates,
flavorings,
preservatives,
and mixtures thereof.

The final pharmaceutical form, as a tablet or capsule, can be coated according to the techniques and formulations known to those skilled in the art in order to improve its presentation: color, appearance, taste masking, etc.

The novel AP-based pharmaceutical forms according to the invention are original in their structure, presentation and composition and can be administered orally, especially in single daily doses.

It may be advantageous to mix, in one and the same capsule, one and the same tablet or one and the same powder for a suspension to be taken orally, at least two types of microparticles with different AP release kinetics, e.g. with immediate release and modified release. It may also be advantageous to mix two (or more) types of microparticles each containing a different AP released according to its own release profile.

Thus the present invention relates especially to a multimicroparticulate pharmaceutical form, characterized in that it contains a plurality of populations of microparticles, said populations differing from one another at least in the nature of the AP present and/or the composition of the coating and/or the thickness of the coating and/or the location of the agent D.

The present invention further relates to a multimicroparticulate pharmaceutical form comprising at least two types of microparticles with different AP release kinetics, e.g. with immediate release and modified release or else with modified release according to different release kinetics.

The present invention further relates to a multimicroparticulate pharmaceutical form additionally comprising a mixture of several AP, each of them being contained in microparticles having identical or different release kinetics.

Without implying a limitation, it must nevertheless be emphasized that the pharmaceutical form according to the invention is of particular value in that it can be presented as a single daily oral dose comprising from 100 (one hundred) to 500,000 reservoir microparticles containing AP.

Furthermore, the invention relates to the use of the microparticles as defined above for the preparation of multimicroparticulate oral pharmaceutical or dietetic forms, preferably as tablets, powders for a suspension to be taken orally, or capsules.

Finally, the invention further relates to an improved therapeutic treatment consisting essentially in administering a pharmaceutical form that is safer as regards the risk of dose dumping in the presence of alcohol.

According to another of its features, the invention further relates to the microparticles per se as defined above.

The present invention further relates to the processes for obtaining the pharmaceutical forms according to the invention as defined above, said processes being made up of several steps consisting essentially in:
a) preparing cores (uncoated microparticles) of AP by:
   extrusion/spheronization of AP, optionally with one or more agents D or pharmaceutically acceptable excipients; and/or
   wet granulation of AP, optionally with one or more agents D or pharmaceutically acceptable excipients; and/or
   compaction of AP, optionally with one or more agents D or pharmaceutically acceptable excipients; and/or
   spraying of AP, optionally with one or more agents D or pharmaceutically acceptable excipients, as a dispersion or solution in an aqueous or organic solvent onto an inert support or particles of agent D; and/or
   sieving of powder or crystals of AP;
b) preparing reservoir microparticles of AP by:
   spraying, in a fluidized air bed, of a solution or dispersion containing one or more compounds A and B and optionally one or more compounds C and/or D onto the microparticles of AP; the microparticles of AP may have been coated beforehand with one or more agents D; the coated microparticles of AP can optionally be coated with one or more agents D; and
c) preparing tile final form of the drug by:
   granulation and/or extrusion/spheronization of the reservoir microparticles of AP with an agent D for introduction into capsules or sachets; or
   mixing of reservoir microparticles of AP, optionally with one or more, agents D and pharmaceutically acceptable excipients, to give a tablet; this tablet can optionally be coated in a coating drum with one or more layers containing the agent D and/or pharmaceutically acceptable excipients; or
   introduction of the reservoir microparticles of AP into capsules; the capsules can optionally be coated in a drum or fluidized air bed with one or more agents D and/or pharmaceutically acceptable excipients; or
   introduction of the reservoir microparticles of AP into sachets, optionally with one or more agents (D) and/or pharmaceutically acceptable excipients; or
   introduction of tablets containing reservoir microparticles of AP into capsules, the tablet containing one or more agents D and it being possible for the capsules to be coated with one or more agents D.

These are advantageous general methodologies which enable the forms of the invention to be produced in a simple and economic manner.

The invention can be carried out independently of the solubility of the AP in water. Four classes of AP are defined, according to their solubility, in the Biopharmaceutics Classification System of the US Food and Drug Administration (Amidon G. L. et al. “A theoretical basis for a biopharmaceutics drug classification: the correlation of in vivo drug product dissolution and in vivo bioavailability”, Pharmaceutical Research, 1995, vol. 12, 413-420). AP belonging to these different classes can be used according to the present invention.

The AP contained in the coated microparticles according to the invention is advantageously selected from at least one of the following families of active substances: agents for treating alcohol abuse, agents for treating Alzheimer's disease, anesthetics, agents for treating acromegaly, analgesics, antiasthmatics, agents for treating allergies, anticancer agents, anti-inflammatories, anticoagulants and antithrombotics, anticonvulsants, antiepileptics, antidiabetics, antiemetics, antiglaucoma agents, antihistamines, anti-infectives, antiparkinsonians, anti-cholinergics, antitussives, carbonic anhydrase inhibitors, cardiovascular agents: hypolipemics, antiardythmics, vasodilators, antianginals, antihypertensives, vaso-protectors and cholinesterase inhibitors, agents for treating central nervous system disorders, central nervous system stimulants, contraceptives, fertility promoters, labor inducers and inhibitors, agents for treating cystic fibrosis, dopamine receptor agonists, agents for treating endometriosis, agents for treating erectile dysfunctions, agents for treating fertility, agents for treating gastrointestinal disorders, immuno-modulators and immunosuppressants, agents for treating memory disorders, anti-migraines, muscle relaxants, nucleoside analogs, agents for treating osteoporosis, parasympathomimetics, prostaglandins, psychotherapeutic agents: sedatives, hypnotics, tranquilizers, neuroleptics, anxiolytics, psychostimulants and anti-depressants, agents for dermatological treatments, steroids and hormones, amphetamines, anorexigenics, non-analgesic painkillers, antiepilepties, barbiturates, benzodiazepines, hypnotics, laxatives and psychotropics.

Examples of agents for treating alcohol abuse are chlorazepate, chlordiazepoxide, diazepam, disulfiram, hydroxyzine, naltrexone and salts, esters, hydrates, polymorphs and isomers thereof.

Examples of anesthetics are lidocaine, midazolam and salts, esters, hydrates, polymorphs and isomers thereof.

Examples of analgesics and/or anti-inflammatories are paracetamol, aspirin, buprenorphine, butorphanol, celecoxib, clofenadol, choline, clonidine, codeine, diclofenac, diflunisal, dihydrocodeine, dihydroergotamine, dihydromorphine, ethylmorphine, etodolac, eletriptan, eptazocine, ergotamine, fentanyl, fenoprofen, hyaluronic acid, hydrocodone, hydromorphone, hylan, ibuprofen, indomethacin, ketorolac, ketotifen, levomethadone, levallorphan, levorphanol, lidocaine, mefenamic acid, meloxicam, meperidine, methadone, morphine, nabumetone, nefopam, naloxone, naltrexone, naproxen, naratriptan, nefazodone, normethadone, oxaprozin, oxycodone, oxymorphone, pentazocin, pethidine, phenpyramide, piritramide, piroxicam, propoxyphen, rizatriptan, ketoprofen, sulindac, sumatriptan, tebacone, tilidine, tolmetine, tramadol, zolmitriptan and salts, esters, hydrates, polymorphs and isomers thereof.

Examples of antiasthmatics are ablukast, azelastine, bunaprolast, cinalukast, cromitrile, cromolyn, enofelast, isambxole, ketotifen, levcromakalim, lodoxamide, montelukast, ontazolast, oxarbazole, oxatomide, piriprost potassium, pirolate, pobilukast, edamine, pranlukast, quazolast, repirinast, ritolukast, sulukast, tetrazolast meglumine, tiaramide, tibenelast, tomelukast, tranilast, verlukast, verofylline, zarirlukast and salts, esters, hydrates, polymorphs and isomers thereof.

Examples of anticancer agents are adriamycin, aldesleukin, allopurinol, altretamine, amifostine, anastrozole, asparaginase, betamethasone, bexaroten, bicalutamide, bleomycin, busulfan, capecitabin, carboplatin, cannustine, chlorambucil, cisplatin, cladribine, conjugated estrogen, cortisone, cyclophosphamide, cytarabine, dacarbazine, daunorubicin, dactinomycin, denileukin, dexamethasone, discodermolide, docetaxel, doxorubicin, eloposidem, epirubicin, epoetin, epothilones, estramustine, esterified estrogen, ethynylestradiol, etoposide, exemestane, flavopirdol, fluconazole, fludarabine, fluorouracil, flutamide, floxuridine, gemcitabine, hexamethylmelamine, hydrocortisone, hydroxyurea, ifosfamide, lemiposide, letrozole, leuprolide, levamisole, levothyroxin, lomustine, mechlorethmine, melphalan, mercaptopurine, megestrol, methotrexate, methylprednisolone, methyltestosterone, mithramycin, mitomycin, mitotane, mitoxantrone, mitozolomide, mutamycin, nilutamide, pamidronate, pentostatin, plicamycin, porfimer, prednisolone, procarbazine, semustine, streptozocin, tamoxifen, temozolamide, teniposide, testolactone, thioguanie, tomudex, toireifen, tretinoin, semustine, streptozolocin, verteprofin, vinblastine, vincristine, vindesine, vinorelbine and salts, esters, hydrates, polymorphs and isomers thereof.

Examples of anticoagulants and antithrombotics are warfarin, danaparoid, alprostadil, anagrelide, araob, ataprost, betaprost, camonagrel, cilostazol, clinprost, clopidogrel, cloricromen, dermatan, desirudine, domitroban, drotaverine, epoprostenol, fradafiban, gabexate, iloprost, isbogrel, lamifiban, lefradafiban, lepirudin, levosimendan, lexipafant, melagatran, nafagrel, nafamostat, nizofenone, orbifiban, ozagrel, pamicogrel, quinobendan, sarpognilate, satigrel, simendan, ticlopidine, vapiprost, tirofiban, xemilofiban, Y20811 and salts, esters, hydrates, polymorphs and isomers thereof.

Examples of anticonvulsants are carbamazepine, clonazepam, clorazepine, diazepam, divalproex, ethosuximide, ethotion, felbamate, fosphenytoin, gabapentine, lamotrigine, levetiracetam, lorazepam, mephenytoin, mephobarbital, metharbital, methsuximide, oxcarbazopine, phenobarbital, phenytoin, pregabaline, primidone, tiagabine, topiramate, valproic acid, vigabatrine, zonisamide and salts, ester, hydrates, polymorphs and isomers thereof.

Examples of antidiabetics are acarbose, acetohexamide, carbutamide, chlorpropamide, epaloretat, glibonuride, gliclazide, glimepiride, glipizide, gliquidone, glisoxepide, glyburide, glyhexamide, metformin, miglitol, nateglinide, orlistat, phenbutamide, pioglitazone, repaglinide, rosiglitazone, tolazainde, tolbutsmide, tolcyclamide, tolrestat, troglitazone, voglibose and salts, esters, hydrates, polymorphs and isomers thereof.

Examples of antimetics are alprazolam, benquimamide, benztropine, betahistine, chlorpromazine, dexamethasone, difenidol, dimenhydrinate, diphenhydramine, dolasetron, domperidone, dronabinol, droperidol, gransetron, haloperidol, lorazepam, meclizine, methylprednisolone, metoclopramide, ondansetron, perphenazine, prochlorperazine, promethazine, scopolamine, tributine, triethylperazine, trifluptomazine, trimethobenamide, tropisetron and salts, esters, hydrates, polymorphs and isomers thereof.

Examples of antiglaucoma agents are alprenoxime, dapiprazole, dipivefrine, latanoprost, naboctate, pimabine and salts, esters, hydrates, polymorphs and isomers thereof.

Examples of antihistamines or beta-agonists are acepromazine, acrivastine, activastine, albuterol, alimemazine, antazoline, azelastin, bitolterol, amlexanox, benzydamine, bromphenirime, cetirizine, chlorpheniramine, cimetidine, cinnarizine, clemastine, clofedanol, cycloheptazine, cyprheptadine, difencloxazine, diphenhydramine, dotarizine, ephedrine, epinastine, epinephrine, ethylnorepinephrine, fenpentadiol, fenpoterol, fexofenadine, flurbiprofen, hydroxyzine, isoetherine, isoproterenol, ketorolac, levocetirizine, levomepromazine, loratidine, mequitazine, metaproterenol niaprazine, oxatomide, oxomemazine, phenylephrine, phenylpropanolamine, pirbuterol, promethazine, pseudoephedrine, pyrilamine, ranitidine, salmeterot, terbutaline, terfenadine, tranilast, xanthine derivatives and salts, esters, hydrates, polymorphs and isomers thereof.

Examples of anti-infectives, especially antibiotics, antifungals and anti-virals, are abacavir, acyclovir, albendazole, amantadine, amphotericin, amikacin, amiosalicylic acid, amoxycillin, ampicillin, amprenavir, atovaquine, azithiomycin, aztreonam, cefaclor, cefadroxil, cefazolin, cefdinir, cefexime, cefpodoxime proxetil, cefprozil, ceftibuten, cephalexine, chloroquine, cidofovir, cilastatin, ciprofloxacin, claithromycin, clavulanic acid, clindamycin, dalfopristine, dapsone, delavirdine, demeclocycline, didanosine, doxycycline, efavirenz, enoxacin, erythromycin, ethambutol, ethionamide, famcyclovir, fluconazole, fluctosine, foscamet, ganciclovir, gatifloxacin, griseofulvin, hydroxychloroquine, indinavir, isoniazide, itraconazole, ivermectil, ketoconazole, lamivudine, levofloxacin, linizolide, lomefloxacin, loracarbef, mebendazole, mefloquine, methanamine, metronidazole, minocycline, moxefloxacin, nalidixic acid, nelfinavir, neomycin, nevirapine, nitrofurantoin, norfloxacin, ofloxacin, oseltamivir, oxytetracycline, penicillin V, perfloxacin, praziquantel, pyrznamide, pyrimediamine, quinidine, quinupristine, retonavir, ribavirin, rifabutin, rifampicin, rimantadine, saquinavir, sparfloxacin, stavudine, streptomycin, sulfamethoxazole, tetramycin, terbinafine, tetracycline, thiabendazole, tobramycin, trinmethoprinm, troleandomycin, trovafloxacin, valacyclovir, vancomycin, zalcitabine, zanamivir, zidovudine and salts, esters, hydrates, polymorphs and isomers thereof.

Examples of antiparkinsonians are amantadine, adrogolide, altinicline, benzatropine, biperiden, brasofensine, bromocriptine, budipine, cabergoline, CHF-1301, dihydrexidine, entacapone, etilevodopa, idazoxane, iometopane, lazabemide, melevodopa, carbidopa, levodopa, mofegiline, moxiraprine, pergolide, pramipexole, quinelorane, rasagiline, ropinirole, seligiline, talipexole, tolcapone, trihexyphenidyl and salts, esters, hydrates, polymorphs and isomers thereof.

Examples of antirheumatics are azathioprine, betamethasone, celecoxib, cyclosporin, diclofenac, hydroxychmoroquine, indomethacin, mercaptobutanedioic acid, methylprednisolone, naproxen, penicillamine, piroxicam, prednisolone, sulfasalazine and salts, esters, hydrates, polymorphs and isomers thereof.

Examples of platelet aggregation inhibitors are anagrelide, aspirin, cilostazol, clopidogrel, dipyridamole, epoprostenol, eptifibatide, ticlopidine, tinofiban and salts, esters, hydrates, polymorphs and isomers thereof

Examples of antispasmodics and anticholinergics are aspirin, atropine, diclofenac, hyoscyamine, mesoprostol, methocaramol phenobarbital, scopolamine and salts, esters, hydrates, polymorphs and isomers thereof.

Examples of antitussives are, parazetamol, acnivastine, benzonatate, beractant, brompheniramine, caffeine, calfactant, carbetapetane, chlorpheniramine, codeine, colfuscerine, dextromethorphan, doxylamine, fexofenadine, guaphenesine, metaproterenol, montelukast pentoxiphylline, phenylephrine, phenylproparolamine, pirbuterol, pseudoephedrine, pyrilamine, terbutaline, theophylline, zafirlukast, zileuton and salts, esters, hydrates, polymorphs and isomers thereof.

Examples of carbonic anhydrase inhibitors are acetazolamide, dichlorphenamide, dorzolamide, methazolamide, sezolamide and salts, esters, hydrates, polymorphs and isomers thereof.

Examples of cardiovascular agents, especially hypolipemics, anti-arrythmics, vasodilators, antianginals, antihypertensives and vasoprotectors, are acebutolol, adenosine, amidarone, amiloride, amlodipine, amyl nitrate, atenolol, atorvastatin, benzepril, bepiridil, betaxalol, bisoprolol, candesartan, captopril, cartenolol, carvedilol, cerivastatin, chorthalidone, chlorthmiazole, clofibrate, clonidine, colestipol, colosevelam, digoxin, diltiazem, disopyramide, dobutamine, dofetilide, doxazosin, enalapiril, epoprostenol, eprosartan, esmolol, ethacirynate, erythrityl, felodipine, fenoidapam, fosinopril, flecainide, furosemide, fluvastatin, gemfibrozil, hydrochlorthiazide, hydroflumethiazine, ibutilide, indapamide, isosorbide, irbesartan, labetolol, lacidipine, lisinopril, losartan, lovastatin, mecamylamine, metoprolol, metaminol, metazolone, methylchlothiazide, methyldopa, metyrosine, mexiletine, midrodine, milrinone, moexipril, nadolol, niacin, nicardipine, nicorandil, nifedipine, nimodipine, nisoldipine, nitroglycerin, phenoxybenzamin perindopril, polythiazide, pravastatin, prazosin, procainamide, propafenone, propranolol, quanfacin, quinapril, quinidine, ranipril, simvastatin, sotalol, spironolactone, telmisartan, terazosin, timolol, tocainamide, torsemide, trandolapril, triamterene, trapidil, valsartan and salts, esters, hydrates, polymorphs and isomers thereof.

Examples of vasodilators are adenosine, alverine, caffeine, dihydroergocomine, enalapril, enoximone, iloprost, kalleone, lidoflazine, nicardipine, nimodipine, nicotinic acid, papaverine, pilocarpine, salbutamol, theophytline, trandolapril, uradipil, vincamine and salts, esters, hydrates, polymorphs and isomers thereof.

Examples of cholinesterase inhibitors are donepezil, neostigmine, pyridostigmine, rivastigmine, tacrine and salts, esters, hydrates, polymorphs and isomers thereof.

Examples of central nervous system stimulants arc caffeine, doxapram, dexoamphetamine, donepezil, methamphetamine, methylphenidate, modafinil, neostigmine, pemoline, phentermine, pyndostigmine, rivastigmine, tacrine and salts, esters, hydrates, polymorphs and isomers thereof.

Examples of contraceptives are desogestral, ethynylestradiol, ethynodiol, levonorgestrel, medroxyprogesterone, mestranol, norgestimate, norethindrone, norgestrel and salts, esters, hydrates, polymorphs and isomers thereof.

Examples of agents for treating cystic fibrosis are pancrelipase, tobramycin and salts, esters, hydrates, polymorphs and isomers thereof.

Examples of dopamine receptor agonists are amantadine, cabergoline, fenoldopam, pergolide, pramipezal, ropinirole and salts, esters, hydrates, polymorphs and isomers thereof.

Examples of agents for treating endometriosis are danazol norethindrone and salts, esters, hydrates, polymorphs and isomers thereof.

Examples of agents for treating erectile dysfunctions are sildenafil, tadalafil vardenafil, yohimbine and salts, esters, hydrates, polymorphs and isomers thereof.

Examples of agents for treating fertility are clomiphene, progesterone and salts, esters, hydrates, polymorphs and isomers thereof.

Examples of agents for treating gastrointestinal disorders are alosetron, bisacodyl, bismuth subsalicylate, celecoxib, cimetidine, difoxine, diphenoxylate, docusate, esomeprazole, famotidine, glycopyrrolate, lansoprazole, loperamide, metoclopramide, nizatidine, omeprazole, pantoprazole, rabeprazole, ranitidine, simethicone, sucralfate and salts, esters, hydrates, polymorphs and isomers thereof.

Examples of immunomodulators and immunosuppressants are azathioprine, ceftizoxime, cyclosporin, leflunomide, levamisol, mycophenolate, phthalidomide, ribavirin, sirolimus, tacrolimus and salts, esters, hydrates, polymorphs and isomers thereof.

Examples of agents for treating Alzheimer's disease are CP 118954, donepezil, galanthamine, metrifonate, revastigmine, tacrine, TAK-147 and salts, esters, hydrates, polymorphs and isomers thereof.

Examples of antimigraines are paracetamol, dihydroergotamine, divalproex, ergotamine, propranolol, risatriptan, sumatriptan, trimetrexate and salts, esters, hydrates, polymorphs and isomers thereof.

Examples of muscle relaxants are azapropazone, baclofen, carisoprodol, quinine derivatives, chloromezanone, chlorphenesin carbamate, chlorozoxazone, cyclobenzaprin, dantrolene, dimethyltabocurarinium chloride, fenyramidol, guaiphenesin, memantin, mephenesin, meprobamate, metamisol, metaxalone, methocarbamol, orphenadrine, phenazone, phenprobamate, tetrazepam, tizanidine, tybamate and salts, esters, hydrates, polymorphs and isomers thereof.

Examples of nucleoside analogs are abacavir, acyclovir, didanosine, gamciclovir, gemcitabine, lamivudine, ribavirin, stavudine, zalcitabine and salts, esters, hydrates, polymorphs and isomers thereof.

Examples of agents for treating osteoporosis are alendronate, calcitonin, estradiol, estropipate, medroxyprogesterone, norethindrone, norgestimate, pamidronate, raloxifen, risdronate, zoledronate and salts, esters, hydrates, polymorphs and isomers thereof.

Examples of parasympathomimetics are bethanechol, biperidine, edrophonium, glycopyrrolate, hyosycyamine, pilocarpine, tacrine, yohimbine and salts, esters, hydrates, polymorphs and isomers thereof.

Examples of prostaglandins are alprostadil, epoprostenol, misoprostol and salts, esters, hydrates, polymorphs and isomers thereof.

Examples of psychotherapeutic agents are acetophenazine, alentemol, alpertine, alprazolam, amitriptyline, apripmazole, azaperone, batelapine, befipiride, benperidol, benzindopyrine, bimithil, biriperone, brofoxine, bromperidol, broniperidol, bupropione, buspirone, butaclamol, butaperzine, carphenazine, carvotroline, chlorazepine, chlordiazepoxide, chlorpromazine, chlorprothixen, cinperene, cintriamide, citalopram, clomacran, clonazcpam, clopenthixol, clopimozide, clopipazan, cloroperonec clothiapine, clothixamide, clozapine, cyclophenazine, dapiprazole, dapoxetine, desipramine, divalproex, dipyridamole, doxepine, droperidol, duloxetine, eltoprazine, eptipirone, etazolate, fenimide, flibanserine, flucindole, flumezapine, fluoxetine, fluphenazine, fluspiperone, fluspirilene, flutroline, fluvoxamine, gepirone, gevotroline, halopemide, haloperidol, hydroxyzine, hydroxynortriptyline, iloperidone, imidoline, lamotrigine, loxapine, enperone, mazapine, mephobarbital, meprobamate, mesoridazine, mesoridazine, milnacipran, mirtazepine, metiapine, milenperone, milipertine, molindone, nafadotride, naranol, nefazodone, neflumozide, ocaperidone, odapipatn, olanzapine, oxethiazine, oxiperomide, pagoclone, paliperidone, paroxitene, penfluridol, pentiapine, peiphenazine, phenelzine, pimozide, pinoxepine, pipamperone, piperacetazine, pipotiazine, piquindone, piracetm, pirlindole, pivagabine, pramipexole, prochlorperazine, promazine, quetiapine, reboxetine, remoxipride, risperidone, rimcazole, robolzotan, selegiline, seperidol, sertraline, sertindole, seteptiline, setoperone, spiperone, sunipitrone, tepirindole, thiordazine, thiothixen, tiapride, tioperidone, tiospirone, topiramate, tranycypromine, trifluoperazine, trflperidol, triflupromauzine, trimipramine, venlafaxine, ziprasidone and salts, esters, hydrates, polymorphs and isomers thereof.

Examples of sedatives, hypnotics and tranquilizers are bromazepam, buspirone, clazolam, clobazam, chlorazepate, diazepam, demoxepam, dexmedetomidine, diphenyhydtenine, doxylamine, enciprazine, estrazolam, hydroxyzine, ketazolam, lorazatone, larazepam, loxapine, medazepam, meperidine, methobarbital, midazolam, nabilone, nisobamate, oxazepam, pentobarbital, promethazine, propofol, triazolam, zaleplon, zolpidem and salts, esters, hydrates, polymers and isomers thereof.

Examples of agents for dermatological treatments are acitretin, alclometasone, alitretinoin, betamethasone, calcipotriene, clobetasol, clocortolone, clotrimazole, cyclosporin, desonide, difluorosone, doxepine, eflomithine, finasteride, flurandrenolide, hydrochloroquine, hydroquinone, hydroxyzine, ketoconazole, mafenide, malathion, menobenzone, neostigmine, nystatin, podophyllotoxin, povidone, tazarotene, tretinoin and salts, esters, hydrates, polymorphs and isomers thereof.

Examples of steroids and hormones are alclometasone, betamethasone, citrorelix, clobetasol, clocortolone, cortisones, danazol, desonide, desogestrel, desoximetasone, dexamethasone, diflorasone, estradiol, estrogens, estropipate, ethynylestradiol, fluocinolone, flurandrenolide, fluticasone, halobetasol, hydrocortisone, leuprolide, levonorgestrel, levothyroxin, medroxyprogesterone, methylprednisolone, methyltestosterone, mometasone, norethindrone, norgestrel, oxandrolone, oxymetholone, prednicarbate, prednisolone, progesterone, stanozolol, testosterone and salts, esters, hydrates, polymorphs and isomers thereof

Reference may also be made to the list of AP given on pages 4 to 8 of patent application EP 0 609 961. The AP used belongs e.g. to at least one of the following families of active substances: amphetamines, analgesics, anorexigenics, antalgics, antidepressants, antiepileptics, antimigmaines, antiparkinsonians, antitussives, anixiolytics, barbiturates, benzodiazepines, hypnotics, laxatives, neuroleptics, opiates, psychostimulants, psychotropic agents, sedatives and stimulants. In the case where the AP is an analgesic AP (aAP), it is preferably an opioid.

Even more precisely, the AP used is selected from the following compounds: anileridine, acetorphine, acetylalphamethylfentanyl, acetyldihydrocodeine, acetylnethadol, alfentanil, allylprodine, alphacetylmethadol, alphameprodine, alphaprodine, alphamethadol, alphamethylfentanyl, alpha-methylthio-fentanyl, alphaprodine, anileridine, butorphanol, benzethidine, benzylmorphine, beta-hydroxyfentanyl, beta-hydroxy-methyl-3-fentanyl, betacetylmehadol, betameprodine, betamethadol, betaprodine, bezitramide, buprenorphine, dioxaphetyl butyrate, clonitazene, cyclazocine, cannabis, cetobemidone, clonitazene, codeine, coca, cocaine, codoxine, dezocine, dimenoxadol, dioxaphetyl butyrate, dipipanone, desomorphine, dextromoramide, dextropropoxyphene, diampromide, diethyl-thiambutene, difenoxine, dihydrocodeine, dihydroetorphine, dihydromorphine, dimenoxadol, dimepheptanol, dimethyltbiambutene, diphenoxylate, dipipanone, dronabinol, drotebanol eptazocine, ethoheptazine, ethylmethylthiambutene, ethylmophine, etonitazene, ecgonine, ephedrine, ethylmethylthiambutene, ethylmorphine, etonitazne, etorphine, etoxeridine, fentanyl, firethidine, heroin, hydrocodone, hydromorphinol, hydromorphone, hydroxypethidine, isomethadone, ketobemidone, levallorphan, lofentanil, levomethorphan, levomoramide, levophenacylmorphan, levorphanol, meptazinol, meperidine, metazocine, methadone, methyldesorphine, methyldihydromorphine, methylphenidate, methyl-3-thiofentanyl, methyl-3-fentanyl, metopon, moramide, morpheridine, morphine, myrophine, nabilone, nalbuphine, narceine, nicomorphine, norlevorphanol, normethadone, nalorphine, normorphine, nicocodine, nicodicodine, nicomorphine, noracymethadol, norcodeine, norievorphanol, normethadone, normorphine, norpipanone, opium, oxycodone, oxymorphone, phenadoxone, phenoperidine, promedol, properidine, propirm, propoxyphen, parafluorofentanyl, pentazocine, pethidine, phenampromide, phenazocine, phenomorphan, phenoperidine, pholcodine, piminodine, pintramide, proheptazine, propranolol, properidine, propirai, racemethorphan, racenoramide, racemorphan, remifentanil, sufentanil, tetrahydrocannabinol, thebacone, thebaine, thiofentanyl, tilidine, trimeperidine, tramadol and pharmaceutically acceptable salts, esters, hydrates, polymorphs and isomers thereof, and mixtures thereof.

The following may be mentioned among the anti-inflammatory AP which can be envisaged: celecoxib, ibuprofen, paracetamol, diclofenac, naproxen, benoxaprofen, flurbiprofen, fenoprofen, flubufen, ketoprofen, indoprofen, piroprofen, carprofen, oxaprozine, pramoprofen, muroprofen, trioxaprofen, suprofen, amnineoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam and pharmaceutically acceptable salts, esters, hydrates, polymorphs and isomers thereof, and mixtures thereof.

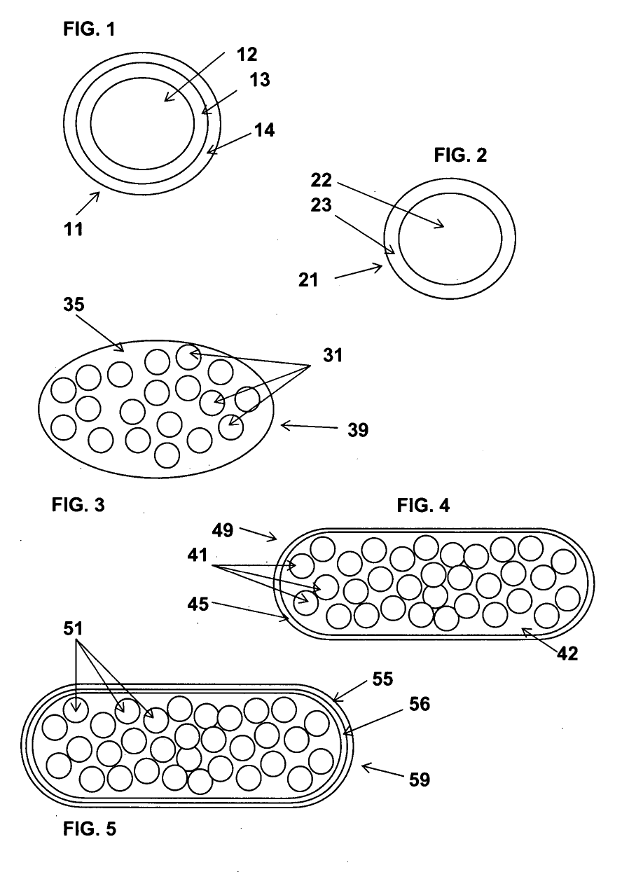

The invention will be explained more clearly by means of the following Examples, which are given solely by way of illustation, afford a good understanding of the invention and make it possible to demonstrate its different embodiments and/or modes of implementation, as well as its different advantages. Various embodiments of the invention are illustrated as non-limiting Examples in FIGS. 1 to 5.

FIG. 1 shows a microparticle 11 whose AP core 12 is covered with a coating 13 on which the agent D 14 is deposited. The coating 13 contains the polymer A, the plasticizer B and optionally the surfactant C.

FIG. 2 shows a microparticle 21 whose AP core 22 contains an agent D1. The AP core 22 is covered with a coating 23, which also contains an agent D2. The agents D1 and D2 can be mutually identical or different

FIG. 3 shows a pellet or granule 39, obtained e.g. by extrusion, which contains microparticles 31 in a binder phase 35 containing at least one agent D. The microparticles 31 comprise reservoir microparticles and optionally uncoated AP microparticles.

FIG. 4 shows a tablet 49 containing microparticles 41 according to the invention, e.g. reservoir microparticles and optionally immediate-release microparticles, in a binder 42 containing an agent D2. The tablet 49 is covered with a coating 45 containing an agent D1. The agents D1 and D2 can be mutually identical or different.

FIG. 5 shows a capsule 59 whose wall 56 is covered with a coating 55 based on an agent D. The capsule 59 contains microparticles 51 according to the invention, e.g. reservoir microparticles and optionally immediate-release microparticles.

### EXAMPLES

### Example 1

### Acyclovir Capsles—the Agent D is Contained in the Inert Support of the Microparticles

Step 1:

288 g of acyclovir and 72 g of hydroxypropyl cellulose (Klucel EF®/Aqualon) are dispersed in 840 g of water. The suspension is sprayed onto 240 g of guar gum (Danisco) in a fluidized air bed (Glatt GPCG1).

1.4 g of ethyl cellulose (Ethocel 20 Premium/Dow), 9.24 g of cellulose acetate-butyrate (CAB 171-15/Eastman), 1.68 g of polysorbate 80 (Tween 80/Uniqema) and 1.68 g of triethyl citrate (Morflex) are solubilized in a mixture composed of 94% of acetone and 6% of water. This solution is sprayed onto 56 g of acyclovir granules (prepared in step 1).

The microparticles obtained are then placed in a size 0 gelatin capsule (to give an acyclovir dose of 150 mg per capsule).

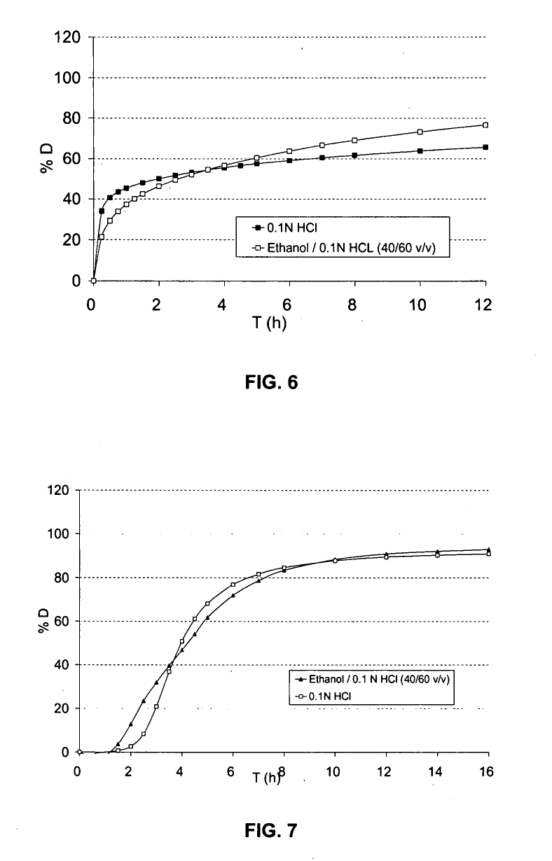

The profiles of dissolution D (%) as a function of time (h) in 900 ml of 0.1 N HCl and in 500 ml of an ethanol/0.1 N HCl mixtur (40/60 v/v), with paddle stirring at 75 rpm, are given in FIG. 6:

It is seen that the dissolution profiles in the media 0.1 N HCl and ethanol/0.1 N HCl (40/60 v/v) are very similar. In particular, there is no substantial acceleration of the amount released in the presence of ethanol (i.e. no dose dumping).

### Example 2

### Metformin Capsule—the Agent D is Contained in the Capsule Coating

Step 1:

500 g of metformin are dispersed in 2586 g of water. The solution is sprayed onto 450 g of cellulose spheres (Asahi-Kasei) in a Glatt GPCG1.

Step 2:

228 g of ethyl cellulose (Ethocel 20 Premium/Dow), 30 g of povidone (Plasdone K29-32/International Specialty Products Inc.), 12 g of polyoxyl-40 hydrogenated castor oil (polyoxyetlylene glycerol trihydroxystearate: Cremophor RH 40/ISP) and 30 g of castor oil are solubilized in a mixture composed of 60% of acetone and 40% of isopropanol. This solution is sprayed onto 700 g of metformin granules prepared in step 1.

The microparticles obtained are then placed in a size 2 gelatin capsule (to give a metformin dose of 150 mg per capsule). This capsule is then film-coated with a solution of sodium carboxymethyl cellulose (Blanose 7 LF/Aqualon) at a rate of 20 mg of sodium carboxymethyl cellulose per 60 mg of gelatin.

The dissolution profiles in 900 ml of 0.1 N HCl and in 500 ml of an ethanol/0.1 N HCl mixture (40/60 v/v), with paddle string at 75 rpm, are given in FIG. 7:

It is seen that the dissolution profiles in the media 0.1 N HCl and ethanol/0.1 N HCl (40/60 v/v) are very similar. In particular, there is no substantial acceleration of the amount released in the presence of ethanol (i.e. no dose dumping).

### Example 3

### Acycovir Capsules—the Agent D is Contained in the Inert Support of the Microparticles and in the Capsule Constituent

Step 1:

288 g of acyclovir and 72 g of hydroxypropyl cellulose (Khicel EF®/Aqualon) are dispersed in 840 g of water. The suspension is sprayed onto 240 g of guar gum (Danisco) in a Glatt GPCG1.

Step 2:

9.84 g of ethyl cellulose (Ethocel 20 Premium/Dow), 0.24 g of povidone (Plasdone K29-32/ISP), 0.24 g of sorbitan monooleate (Span 80/Uniqema) and 1.68 g of castor oil (Oarbit Huilerie) are solubilized in a mixture composed of 60% of acetone and 40% of isopropanol. This solution is sprayed onto 48 g of acyclovir granulets (prepared in step 1).

The microparticles obtained are then placed in a size 0 vegetable capsule (based on hypromellose [or HPMC]) (to give an acyclovir dose of 150 mg per capsule).

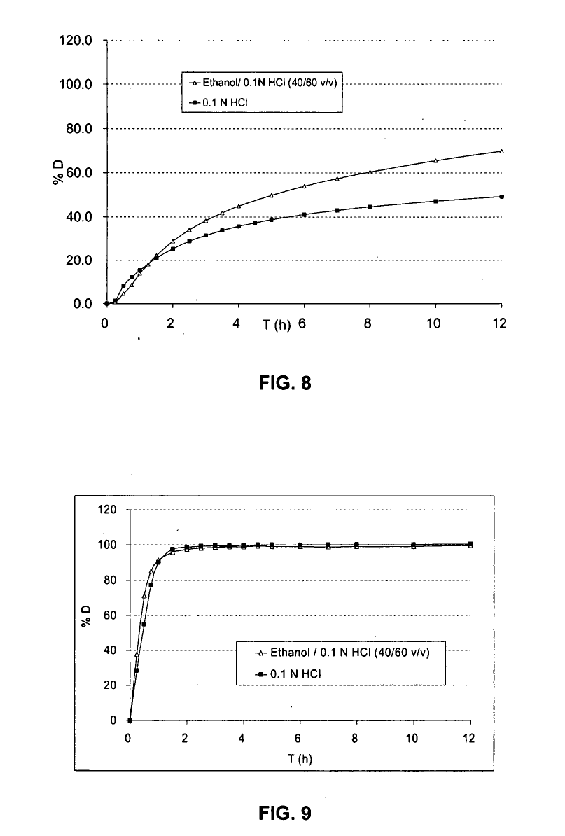

The dissolution profiles in 900 ml of 0.1 N HCl and in 500 ml of an ethanol/0.1 N HCl mixture (40/60 v/v), with paddle stirring at 75 rpm, are given in FIG. 8:

It is seen that the dissolution profiles in the media 0.1 N HCl and ethanol/0.1 N HCl (40/60 v/v) are very similar. In particular, there is no substantial acceleration of the amount released in the presence of ethanol (i.e. no dose dumping).

### Example 4

### Metformin Capsule—the Agent D is Mixed with the Microparticles

Step 1:

350 g of metformin, 50 g of hydroxypropyl cellulose (Klucel EF®/Aqualon) and 100 g of sodium starch glyrolate (Primojet/Avebe) are dispersed in 700 g of water and 467 g of ethanol. The solution is sprayed onto 500 g of guar gum (Danisco) in a Glatt GPCG1.

Step 2:

224 g of ethyl cellulose (Ethocel 20 Premium/Dow), 5.2 g of sorbitan monooleate (Span 80/Uniqema) and 31.2 g of castor oil (Garbit Huilerie) are solubilized in a mixture composed of 60% of acetone and 40% of isoprcpanol. This solution is sprayed onto 390 g of metformin granules (prepared in step 1).

Step 3:

200 g of microparticles obtained at the end of step 2 are mixed with 65 g of mannitol (Pearlitol SD 200), 30 g of hypromellose [or HPMC] (Methocel E5), 5 g of magnesium stearate and approx. 60 g of water and extruded through a 1.5 mm grid (Fitzpatrick MG-55 extruder). The rods obtained are then spheronized on a plate of roughness 1 mm at a speed of 1500 rpm (Fitzpatrick Q-230.T laboratory spheronizer).

The microparticles obtained are then placed in a size 0 gelatin capsule (to give a metformin dose of 80 mg per capsule).

The dissolution profiles in 900 ml of 0.1 N HCl and in 500 ml of an ethanol/0.1 N HCl mixture (40/60 v/v), with paddle stirring at 75 rpm, are given in FIG. 9:

It is seen that the dissolution profiles in the media 0.1 N HCl and ethanol/0.1 N HCl (40/60 v/v) are very similar.

In both cases approx. 75% of the AP is released in 45 min, which represents the limit of MR forms. To slow down the release of the AP further, those skilled in the art may in particular increase the size of the microparticles or increase the coating rate.

### Example 5

### Behavior of Agents D in Aqueous and Alcoholic Solutions

Different compounds D are introduced into a jar containing either water (on the left in the Figures) or an ethanol/water solution in a ratio of 40/60 v/v (right jar in the Figures).

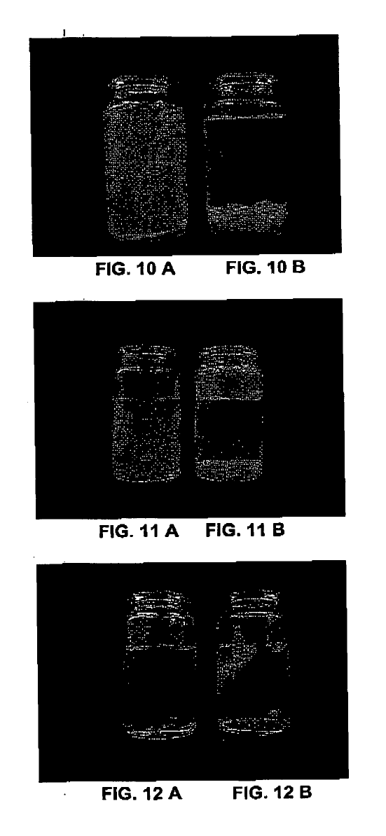

FIG. 10 shows the appearance after 15 min in the case of a substance that is insoluble in water and ethanol—in this case sodium starch glycolate (Primojel®/Avebe)—but which swells more in water than in alcoholic solution.

FIG. 11 shows the case of a substance that is soluble in water but not in the water/ethanol mixture—in this case guar gum (Gridsted® Guar/Danisco).

FIG. 12 shows the appearance after 30 min in the case of a substance whose solubilization rate is higher in water than in the water/ethanol mixture—in this case hypromellose [or HPMC] (Methocel® E5/Dow).

### Example 6

### Metformin Capsule—the Agent D is Mixed with the Microparticles

Step 1:

1700 g of metformin are solubilized in 2348 g of water. The solution is sprayed onto 300 g of cellulose spheres (Cellets 90/Pharmatrans) in a Glatt GPCG1.

Step 2:

249.6 g of ethyl cellulose (Ethocel 20 Premium/Dow), 19.2 g of povidone (Plasdone K29-32/ISP), 12.8 g of polyoxyl-40 hydrogenated castor oil (Cremophor RH 40/BASF) and 38.4 g of castor oil (Garbit Huilerie) are solubilized in a mixture composed of 60% of acetone and 40% of isopropanol. This solution is sprayed onto 480 g of metformin granules (prepared in step 1).

Step 3:

6 g of microparticles obtained in step 2 are mixed with 0.4 g of hypromellose [or HPMC] (Methocel E4M/Colorcon), 0.2 g of hydroxypropyl cellulose (Klucel HF/Aqualon) and 0.04 g of magnesium stearate in a rotating drum mixer (Mini 80/Engelsmann AG) for 30 min. The mixture obtained is then placed in a size 0 gelatin capsule (to give a metformin dose of about 150 mg per capsule).

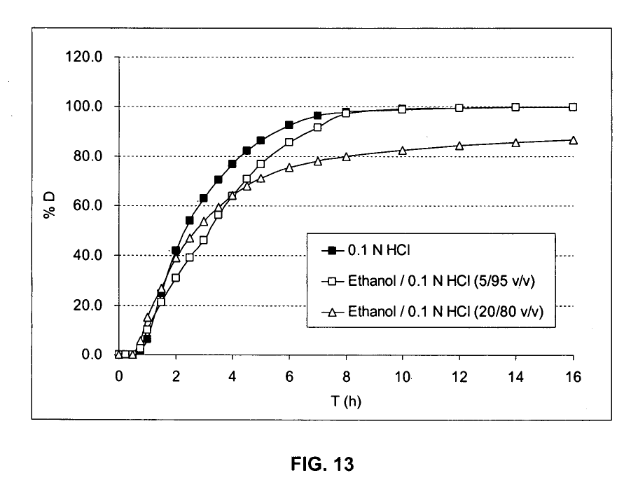

The dissolution profiles in 900 ml of 0.1 N HCl, in 900 ml of an ethanol/0.1 N HCl mixture (5/95 v/v) and in 900 ml of an ethanol/0.1 N HCl mixture (20/80 v/v), with paddle stirring at 75 rpm, are given in FIG. 13:

It is seen that the dissolution profile in the medium 0.1 N HCl is similar to or more rapid than those in the media containing ethanol.

1. Oral pharmaceutical or dietetic form comprising microparticles of the reservoir type for the modified release of at least one active principle (AP), characterized in that it is resistant to immediate dumping of the dose of AP in the presence of alcohol.

2. Form according to claim 1, characterized in that the time taken to release 50% of the AP in alcoholic solution:
is not reduced more than 3-fold relative to the time taken to release 50% of the AP in an alcohol-free aqueous medium;
is preferably not reduced more than 2-fold relative to the time taken to release 50% of the AP in an alcohol-free aqueous medium;
is preferably not reduced more than 1.5-fold relative to the time taken to release 50% of the AP in an alcohol-free aqueous medium;
is preferably similar to the time taken in an alcohol-free aqueous medium according to the similarity factor f₂;
or is longer than the time taken to release 50% of the AP in an alcohol-free aqueous medium.

3. Form according to claim 1 or 2, characterized in that it comprises at least one agent D, which is a pharmaceutically acceptable compound whose hydration or solvation rate or capacity is greater in an alcohol-free aqueous medium than in alcoholic solution.

4. Form according to claim 3, characterized in that it comprises microparticles of the reservoir type:
whose mean diameter is less than 2000 μm, particularly preferably between 50 and 800 μm and very particularly preferably between 100 and 600 μm,
and which individually consist of a core which contains the AP and is covered with a coating comprising:
at least one polymer A that is insoluble in the fluids of the gastrointestinal tract;
at least one plasticizer B;
and optionally at least one surfactant C.

5. Microparticulate form according to claim 3 or 4, characterized in that the agent D is:
one of the constituents of the AP core (or uncoated AP microparticle), i.e.:
in the inert support of the microparticles,
and/or in the layer containing the AP, deposited on the inert support of the microparticles,
and/or in the granules containing the AP;
and/or one of the constituents of the coating of the microparticles;
and/or mixed with the microparticles;
and/or one of the external constituents of a monolithic form.

6. Form according to claim 5, characterized in that the agent D is present in the AP core in an amount of 5% to 70% w/w, preferably of 15% to 60% w/w, based on the total weight of the AP core.

7. Form according to claim 5, characterized in that the agent D is present in the coating in an amount of 3 to 30% w/w, preferably of 10% to 20% w/w, based on the total weight of the coating.

8. Form according to claim 5, chamatereed in that the agent D is present in a mixture with the microparticles in an amount of 2 to 30% w/w, preferably of 5% to 25% w/w and particularly preferably of 5% to 20% w/w, based on the total weight of the mixture.

9. Form according to any one of claims 3 to 8, characterized in that the agent D is selected from the following group of products:
crosslinked carboxyalkyl celluloses: crosslinked carboxymethyl celluloses (e.g. sodium croscarmellose),
polyalkylene oxides (e.g. polyethylene oxide or polypropylene oxide),
(hydroxy)(alkyl) celluloses (e.g. hydroxypropyl cellulose, hypromellose [or HPMC]),
carboxyalkyl celluloses (e.g. carboxymethyl cellulose) and salts thereof,
celluloses (powdered or microcrystalline),
polacrilin potassium,
polysaccharides, e.g.:
native starches (e.g. maize, wheat or potato starch) or modified starches (e.g. modified with sodium glycolate),
alginates and salts thereof such as sodium alginate,
guar gums,
carrageenans,
pullulans,
pectins,
chitosans and derivatives thereof,
and mixtures thereof,
proteins, e.g.:
gelatin,
albumins,
casein,
lactoglobulins,
and mixtures thereof,
clays such as bentonite, laponite,
and mixtures thereof.

10. Multimicroparticulate pharmaceutical form according to any one of claims 3 to 9, characterized in that the agent D is selected from the following group of products:
hydroxyalkyl celluloses (e.g. hydroxypropyl cellulose, hypromellose [or HPMC]),
guar gums,
carrageenans,
pullulans,
and mixtures thereof

11. Pharmaceutical for m according to any one of claims 4 to 10, characterized in that:
the polymer A is present in the coating of the reservoir microparticles in an amount of 70% to 95% w/w, preferably of 75% to 95% w/w and particularly preferably of 80 to 95% of the total weight of the coating excluding agent D,
the plasticizer B is present in the coating of the reservoir microparticles in an amount of 1 to 30% w/w, preferably of 2 to 25% w/w and particularly preferably of 5 to 20% of the total weight of the coating excluding agent D,
and the surfactant C is present in the coating of the reservoir microparticles in an amount of 0 to 30% w/w, preferably of 0 to 20% w/w and particularly preferably of 5 to 15% of the total weight of the coating excluding agent D.

12. Pharmaceutical form according to any one of claims 4 to 11, characterized in that the polymer A is selected from the following group of products:
water-insoluble cellulose derivatives,
(meth)acrylic (co)polymer derivatives,
and mixtures thereof.

13. Pharmaceutical form according to claim 12, characterized in that A is selected from the following group of products: ethyl cellulose, cellulose acetate-butyrate, cellulose acetate, type A and type B ammonio-methacrylate copolymers (Eudragit® RS, Eudragito RL, Eudragit® RS PO, Eudragit® PL PO), poly(meth)-acrylic acid esters (Eudragit® NE 30D) and mixtures thereof, ethyl cellulose and/or cellulose acetate being particularly preferred.

14. Pharmaceutical form according to any one of claims 4 to 13, characterized in that the plasticizer B is selected from the following group of products:
glycerol and esters thereof preferably from the following subgroup: acetylated glycerides, glyceryl monostearate, glyceryl triacetate, glyceryl tributyrate,
phthalates, preferably from the following subgroup: dibutyl phthalate, diethyl phthalate, dimethyl phthalate, dioctyl phthalate,
citrates, preferably from the following subgroup: acetylcitric acid tributyl ester, acetylcitric acid triethyl ester, tributyl citxate, triethyl citrate,
sebactes, preferably from the following subgroup: diethyl sebacate, dibutyl sebacate,
adipates,
azelates,
benzoates,
chlorobutanol,
polyethylene glycols,
vegetable oils,
fumarates, preferably diethyl fumarate,
malates, preferably diethyl malate,
oxalates, preferably diethyl oxalate,
succinates, preferably dibutyl succinate,
butyrates,
cetyl alcohol esters,
malonates, preferably diethyl malonate,
castor oil (this being particularly preferred),
and mixture thereof

15. Pharmaceutical form according to any one of claims 4 to 14, characterized in that the surfactant C is selected from the following group of products:
alkali metal or alkaline earth metal salts of fatty acids, sodium dodecylsulfate and sodium docusate being preferred,
polyoxyethylenated oils, preferably polyoxyethylenated hydrogenated castor oil,
polyoxyethylene/polyoxypropylene copolymers,
polyoxyethylenated sorbitan esters,
polyoxyethylenated castor oil derivatives,
stearates, preferably calcium, magnesium, aluminum or zinc stearate,
polysorbates,
stearylfumarates, preferably sodium stearylfumarates,
glycerol behenate,
benzalkonium chloride,
acetyltrimethylammonium bromide,
and mixture thereof.

16. Pharmaceutical form according to claim 4, characterized in that:
the polymer A is ethyl cellulose;
the plasticizer B is castor oil;
the surfactant C is polysorbate;
and the agent D is selected from guar gum, hypromellose [or HPMC], sodium carboxymethyl cellulose, pullulan, sodium starch glycolate and mixtures thereof.

17. Pharmaceutical form according to any one of claims 3 to 16, characterized in that it contains extruded particles, the particles comprising:
microparticles of the reservoir type for the modified release of at least one AP, and
at least one agent D, the agent D representing from 5 to 20% w/w of the microparticles.

18. Pharmaceutical form according to any one of claims 3 to 16, characterized in that it is a capsule; preferably a gelatin-based capsule, which:
is covered with a coating based on sodium carboxymethyl cellulose in an amount of 25% w/w of sodium carboxymethyl cellulose, based on the weight of the empty capsules,
and contains reservoir microparticles.

19. Pharmaceutical form according to any one of claims 3 to 16, characterized in that it is a capsule based on an agent D, preferably based on pullulan.

20. Pharmaceutical form according to any one of claims 3 to 16, characterized in that it is a capsule based on an agent D, preferably based on hypromellose [or HPMC].

21. Pharmaceutical form according to any one of claims 3 to 19, characterized in that it contains a plurality of populations of microparticles, said populations differing from one another at least in the nature of the AP present and/or the composition of the coating and/or the thickness of the coating and/or the location of the agent D.

22. Pharmaceutical form according to claim 20 comprising at least two types of microparticles with different AP release kinetics, e.g. with immediate release and modified release or else with modified release according to different release kinetics.

23. Pharmaceutical form according to claim 20 or 21 additionally comprising a mixture of several AP, each of them being contained in microparticles having identical or different release kinetics.

24. Process for obtaining a pharmaceutical form according to any one of the preceding claims, in several steps consisting essentially in:
a) preparing cores (uncoated microparticles) of AP by:
extrusion/spheronization of AP, optionally with one or more agents D or pharmaceutically acceptable excipients; and/or
wet granulation of AP, optionally with one or more agents D or pharmaceutically acceptable excipients; and/or
compaction of AP, optionally with one or more agents D or pharmaceutically acceptable excipients; and/or
spraying of AP, optionally with one or more agents D or pharmaceutically acceptable excipients, as a dispersion or solution in an aqueous or organic solvent onto an inert support or particles of agent D; and/or
sieving of powder or crystals of AP;
b) preparing reservoir microparticles of AP by:
spraying, in a fluidized air bed, of a solution or dispersion containing one or more compounds A and B and optionally one or more compounds C and/or D onto the microparticles of AP; the microparticles of AP may have been coated beforehand with one or more agents D; the coated microparticles of AP can optionally be coated with one or more agents D; and
c) preparing the final form of the drug by.
granulation and/or extrusion/spheronization of the reservoir microparticles of AP with an agent D for introduction into capsules or sachets; or
mixing of reservoir microparticles of AP, optionally with one or more agents D and pharmaceutically acceptable excipients, to give a tablet; this tablet can optionally be coated in a coating drum with one or more layers containing the agent D and/or pharmaceutically acceptable excipients; or
introduction of the reservoir microparticles of AP into capsules; the capsules can optionally be coated in a drum or fluidized air bed with one or more agents D and/or pharmaceutically acceptable excipients; or
introduction of the reservoir microparticles of AP into sachets, optionally with one or more agents D and/or pharmaceutically acceptable excipients; or
introduction of tablets containing reservoir microparticles of AP into capsules, the tablet containing one or more agents D and it being possible for the capsules to be coated with one or more agents D.
